SEQUENCE LISTING (7 sequences)

SEQ ID NO: 1 - DNA, 400 bp, artificial
  TGFbeta1 including 64 base pair insert
cccacccaccccagccaccccagccctgcagaggcggggcctccgcatcccacctttccgcccccccaccccggggttcgcgctctccgccgggccgcgcgcgcgagcgagcgagcgcggggcgccgcctctcccatgccgccctcggggctgcggctcctgccgcttctgctcccgctgccgtggctcctggtgctgacgccggggaggccagccgtggggctctccacctgcaagaccatcgacatggagggcagagacctgtttctctgcttcttgggaagctgaggcaggagaatttcaagttttagggcagctggtgaaacgcaaacgcatcgaagccatccgtggccagatcctgtccaaactccggctcgccagccccccgagccagggggag

SEQ ID NO: 2 - DNA, 400 bp, artificial
  TGFbeta1 including 64 base pair insert
ctccccctggctcggggggctggcgagccggagtttggacaggatctggccacggatggcttcgatgcgtttgcgtttcaccagctgccctaaaacttgaaattctcctgcctcagcttcccaagaagcagagaaacaggtctctgccctccatgtcgatggtcttgcaggtggagagccccacggctggcctccccggcgtcagcaccaggagccacggcagcgggagcagaagcggcaggagccgcagccccgagggcggcatgggagaggcggcgccccgcgctcgctcgctcgcgcgcgcggcccggcggagagcgcgaaccccggggtgggggggcggaaaggtgggatgcggaggccccgcctctgcagggctggggtggctggggtgggtggg

SEQ ID NO: 3 - protein, 50 aa, artificial
  Truncated TGFbeta1
MPPSGLRLLPLLLPLPWLLVLTPGRPAVGLSTCKTIDMEGRDLFLCFLGS

SEQ ID NO: 4 - DNA, 20 bp, artificial
  primer
gctcccctatttaagaacac

SEQ ID NO: 5 - DNA, 20 bp, artificial
  primer
gctctgccggtggtttcctc

SEQ ID NO: 6 - DNA, 20 bp, artificial
  primer
ttcagggctctctcctaacc

SEQ ID NO: 7 - protein, 37 aa, artificial
  TGFbeta1 downstream of stop codon
GRRISSFRAAGETQTHRSHPWPDPVQTPARQPPEPGG

(19) United States
(12) Patent Application Publication
(10) Pub. No.: US 2023/0143582 A1
(43) Pub. Date: May 11, 2023

(54) METHODS OF USING A TGF-beta KNOCKOUT CELL LINE AND COMPOSITIONS RESULTING THEREFROM

(71) Applicant: BIO-TECHNE CORPORATION, Minneapolis, MN (US)

(72) Inventors: Anthony D. Person, Minneapolis, MN (US); John E. Humphrey, Minneapolis, MN (US)

(21) Appl. No.: 17/797,496

(22) PCT Filed: Mar. 23, 2021
(86) PCT No.: PCT/US21/23710

§ 371 Date: Aug. 4, 2022

Related U.S. Application Data
(60) Provisional application No. 62/994,004, filed on Mar. 24, 2020.

(51) Int. Cl. C07K 14/475; C12N 5/00; C12N 15/90; C07K 14/51

(52) U.S. Cl. CPC C07K 14/475; C12N 5/00; C12N 15/907; C07K 14/51

(57) ABSTRACT

This disclosure describes protein compositions including minimal TGFβ1 contamination, methods for making those protein compositions, and methods for using those protein compositions. In one aspect, this disclosure describes protein compositions produced by a TGFβ1 knockout cell line and methods for using those compositions including, for example, in research or as a therapeutic or prophylactic. In another aspect, this disclosure describes a TGFβ1 knockout cell line and methods of making that cell line.

### CROSS-REFERENCE TO RELATED APPLICATIONS

This application is a § 371 National Stage of International Application No. PCT/US2021/023710 filed Mar. 23, 2021, which claims the benefit of U.S. Provisional Application Ser. No. 62/994,004, filed Mar. 24, 2020, each of which is incorporated by reference herein.

### SEQUENCE LISTING

This application contains a Sequence Listing electronically submitted to the United States Patent and Trademark Office via EFS-Web as an ASCII text file entitled “0541_000008WO01_ST25.txt” having a size of 4 kilobytes and created on Dec. 13, 2022. Due to the electronic filing of the Sequence Listing, the electronically submitted Sequence Listing serves as both the paper copy required by 37 CFR § 1.821(c) and the computer readable form (CRF) required by § 1.821(e). The information contained in the Sequence Listing is incorporated by reference herein.

### SUMMARY OF THE INVENTION

In one aspect, this disclosure describes a method for producing a target biologic, the method including culturing a TGFβ1 knockout cell line that overexpresses the target biologic and purifying the target biologic. As used herein, a “biologic” refers to a product that is produced from a living organism or contains a component of living organism. Exemplary biologics may include, for example, a recombinant protein (including, for example, a monoclonal antibody, a glycoprotein, a peptide hormone, or a toxin), a ribonucleoprotein, a non-peptide hormone (including, for example, a steroid hormone or an eicosanoids hormone), a glycolipid, a subcellular organelle, a blood component, etc. Combinations of the exemplary biologics are also contemplated including, for example, a recombinant glycoprotein or a subcellular organelle isolated from a blood component.

In another aspect, this disclosure describes compositions including a target biologic produced by a method as disclosed herein.

In a further aspect, this disclosure describes a composition that includes a target biologic, wherein the composition includes less than 0.5 nanogram per milliliter (ng/mL) TGFβ1 protein, less than 0.1 ng/mL TGFβ1 protein, less than 0.01 ng/mL TGFβ1 protein, less than 0.05 ng/mL (50 picogram per milliliter (pg/mL)) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 femtogram per milliliter (fg/mL)) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.

In some embodiments, the target biologic includes a TGFβ superfamily ligand. In some embodiments, the target biologic includes an antibody. In some embodiments, the target biologic includes a biologic drug, that is, a biologic used for treatment of a disease or condition in a subject.

As used herein, a “recombinant protein” refers to a protein resulting from the expression of recombinant DNA.

As used herein, “antibody” includes polyclonal antibodies; monoclonal antibodies; antibody fragments, also referred to as antigen binding fragments; and single domain antibodies including antibodies from camelids (for example, camels and llamas) and cartilaginous fish (for example, wobbegong and nurse sharks). Examples of antibody fragments include, for example, Fab, Fab′, Fd, Fd′, Fv, dAB, and F(ab′)2 fragments produced by proteolytic digestion and/or reducing disulfide bridges and fragments produced from a Fab expression library.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows the results of an exemplary genomic PCR, demonstrating a genetic mutation in the TGFβ1 genomic sequence of a CHO-S TGFβ1 knockout (KO) PCR product. Sample 1 is a CHO-S wild type (wt) PCR product; Sample 2 is a CHO-S TGFβ1 KO PCR product. The PCR amplicon from the CHO-S TGFβ1 KO DNA (Sample 2) exhibited a band approximately 64-base pairs larger than the PCR amplicon from wild type CHO-S genomic DNA (Sample 1).

FIG. 2 shows the verification of the introduction of a 64-base pair (bp) insert (indicated by brackets) into the TGFβ1 genomic sequence of the CHO-S TGFβ1 KO cells by DNA sequencing. A stop codon at amino acid 51 (shown by a “-” and highlighted in gray) was introduced by insertions and deletions (INDELs) by the error prone non-homologous end joining (NHEJ) double strand repair mechanism. The positive strand (shown 5′ to 3′) of the DNA sequence is SEQ ID NO:1. The negative strand (shown 3′ to 5′) of the DNA sequence is SEQ ID NO:2. The truncated TGFβ1 amino acid sequence is SEQ ID NO:3. The TGFβ1 amino acid sequence downstream of the stop codon is SEQ ID NO:7.

FIG. 3 shows exemplary ELISA results indicating that a TGFβ1 KO CHO cell line (CHO-S TGFβ1 KO) produces a significantly reduced amount of TGFβ1, as measured by enzyme-linked immunosorbent assay (ELISA), compared to the wild type CHO cell line (CHO-S wt). Detectable amounts of TGFβ1 were present in the conditioned media of CHO-S wt cells, HEK293 wt, and HEK293 cells expressing recombinant hGDF-15 (HEK wt+hGDF-15). The amount of TGFβ1 present in the conditioned media of CHO-S TFGβ1 knockout samples (CHO-S TGFβ KO) was below the detectable range. TGFβ1 levels were also below the detection limit in stable CHO-S TGFβ1 KO cells expressing recombinant hGDF-15 (CHO-S TGFβ1 KO+hGDF-15). Quantification of the results of FIG. 3 is shown in Table 1. Table 1 also shows that CHO-S TGFβ1 KO cells expressing recombinant hTGFB1 (CHO-S TGFβ1 KO+hTGFβ1) had significantly higher levels of TGFB1 than the parental cell line. In FIG. 3 and Table 1, “*” indicates the measured value was below the detection limit of the assay.

FIG. 4A shows recombinant hGDF15 protein that includes higher levels of TGFβ1 contamination can cause activation of Smad2 in the DU145 human prostate cancer cell line. The contents of each lane of FIG. 4A are described in Table 2A. FIG. 4B shows recombinant hGDF15 made in CHO-S TGFβ1 knockout cells does not activate phosphorylation of SMAD-2 when used to treat DU145 cells. The contents of each lane of FIG. 4B are described in Table 2B. Recombinant human TGFβ1 protein alone (1 ng/ml, lane 2), recombinant human GDF-15 from HEK293 cells (2 μg/ml, lane 6), and recombinant human GDF-15 from CHO-S wild type cells (2 μg/ml, lane 8) all induce phosphorylation of SMAD-2. The phosphorylation of SMAD-2 can be rescued with Chicken anti-TGFβ 1 antibody treatment demonstrating that this phosphorylation of SMAD-2 is TGFβ1 dependent. Recombinant human GDF-15 produced in TGFβ1 KO CHO-S cell line does not show any SMAD-2 phosphorylation in DU145 cells at a 2 μg/ml dose, demonstrating removal of the TGFβ1 protein contamination (lane 4).

FIG. 5 shows TGFβ1 negatively affects osteoblast differentiation. MC3T3-E1 preosteoblasts were treated with recombinant mouse Wnt-3a protein from a lot known to have a relatively high level of TGFβ1 contamination (78 picograms (pg) of TGFβ1 protein per microgram (μg) of Wnt-3a protein) (circles). The same dose of mouse Wnt-3a protein was added to MC3T3-E1 cells in the presence of a saturating dose (50 μg/mL) of a chicken anti-TGFβ 1 blocking antibody (triangles). Osteoblast differentiation was measured by quantifying intracellular alkaline phosphatase enzyme activity after three days of treatment.

FIG. 6 shows TGFβ1 negatively affects Wnt-3a-induced osteoblast differentiation. MC3T3-E1 preosteoblasts treated with recombinant human Wnt-3a protein exhibited a dose-responsive induction of osteoblast differentiation, signified by increasing alkaline phosphatase activity three days after addition of the Wnt-3a protein. The median effective dose (ED50) for this effect was 1.78 ng/mL (circles). When a constant dose of 20 ng/mL of recombinant human Wnt-3a protein was added to MC3T3-E1 cells for three days, a relatively high level of alkaline phosphatase activity resulted (flat line and triangles). If the same steady dose of 20 ng/mL of recombinant human Wnt-3a protein was added to cells and a dose titration of recombinant human TGFβ1 was added to this steady 20 ng/mL dose of Human Wnt-3a protein (squares), inhibition of Wnt-3a-mediated osteoblast differentiation was observed at a neutralizing dose of 50 percent (%) (ND50) of 53.3 femtogram per milliliter (fg/mL) TGFβ1.

FIG. 7 shows that the hWnt3a proteins, with relatively higher levels of TGFβ1 protein (see Table 3), show inhibition of MC3T3-E1 differentiation at the highest doses of hWnt3a protein, while the hWnt3a protein purified from the CHO-S TGFβ1 KO line do not show this inhibition of differentiation at higher hWnt3a doses. Lot #DLGC02 from a CHO-S TGFβ1 KO line (circles) shows a sigmoidal curve with a dose responsive increase in osteoblast differentiation assayed by alkaline phosphatase activity assays. The top three doses of both CHO-S wt-derived lots (RSK51 (squares) and RSK69 (triangles)) show significant reduction in osteoinductive activity in the highest three hWnt3a doses (1.67 ug/ml, 0.556 ug/ml, and 0.185 ug/ml), indicative of TGFβ1 inhibitory activity. This “dip” in activity was not observed when MC3T3-E1 cells were treated at the same doses with lot #DGLC02 (hWnt3a protein derived from the CHO-S TGFβ1 KO line) (circles).

FIG. 8 shows that hWnt3a proteins with high or low levels of TGFβ1 protein show similar activity in a HEK293 TCF9-Secreted Alkaline Phosphatase (SEAP) Wnt-responsive reporter assay. hWnt3a proteins from CHO-S TGFβ1 KO cells (lot #DLGC02, circles), CHO-S wt (lot #RSK51, squares), and CHO-S wt (lot #RSK69, triangles) all showed comparable activity. The ED50 for these lots: RSK51=145 ng/ml, RSK69=191 ng/ml, and DLGC02=221 ng/ml.

FIG. 9 shows that hWnt3a protein with high levels of TGFβ1 contamination results in lower osteoinductive activity at high hWnt3a doses in a MC3T3-E1 assay than hWnt3a protein purified from TGFβ1 KO CHO-S cells. The dose-response curve from hWnt3a purified from wild type CHO-S cells (hWnt3a, lot RSK 51, circles) showed a pronounced reduction in activity at the highest four doses of hWnt3a. When the same doses of hWnt3a from the same lot were added to the MC3T3-E1 cells in the presence of a static dose of 10 μg/ml of the TGFβ1 blocking antibody (hWnt3a, lot RSK51+10 μg/ml of TGFβ1 blocking antibody, triangles), the effect of high dose hWnt3a treatment doses was “rescued” to higher levels. hWnt3a protein purified from TGFβ1 KO CHO-S cells (hWnt3a, lot DLGC02, squares) which is known to have a lower level of TGFβ1 contamination (4.97 pg of TGFβ1/μg of hWnt3a, see Table 3) showed a much less pronounced drop in activity at high hWnt3a doses than hWnt3a purified from wild type CHO-S cells (hWnt3a, lot RSK51, diamonds) which has a much higher level of TGFβ1 (114.6 pg of TGFβ1/μg of hWnt3a, see Table 3.) The low level of TGFβ1 levels in purified hWnt3a lot DLG02 (squares) could be further rescued to higher activity levels with addition of 10 μg/ml of TGF1 blocking antibody (triangles) showing that even relatively lower levels of TGFβ1 contamination present in the serum used in the cell culture production media can result in unwanted off-target effects in osteoinductive cellular bioassays.

### DETAILED DESCRIPTION OF ILLUSTRATIVE EMBODIMENTS

This disclosure describes compositions including a target biologic and minimal TGFβ1 contamination, methods for making those compositions, and methods for using those compositions. In some aspects, a method for making the composition includes culturing a TGFβ1 knockout cell line that overexpresses the target biologic and purifying the target biologic.

On occasion, endogenous proteins produced by host cell lines may be copurified with a target protein produced by the cell line. If the endogenous protein has very potent bioactivity, even a very low level of contamination may negatively affect the performance of the product. Chinese Hamster Ovary (CHO) cells are often used by pharmaceutical and biotech companies for the manufacturing of recombinant antibodies and other proteins.

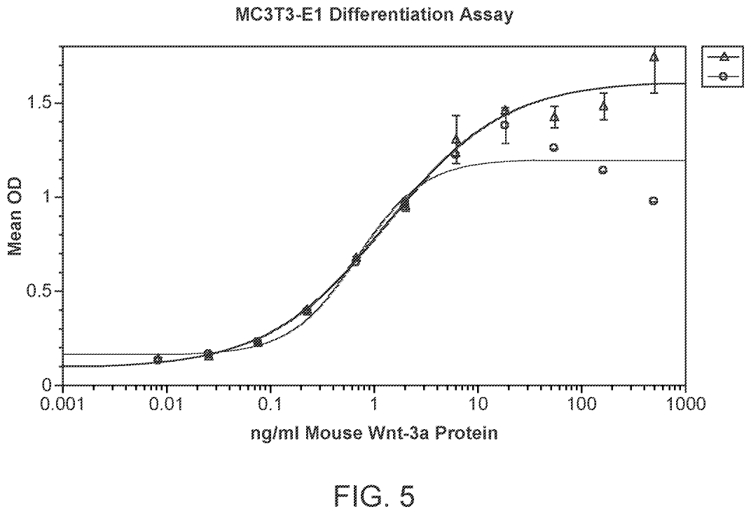

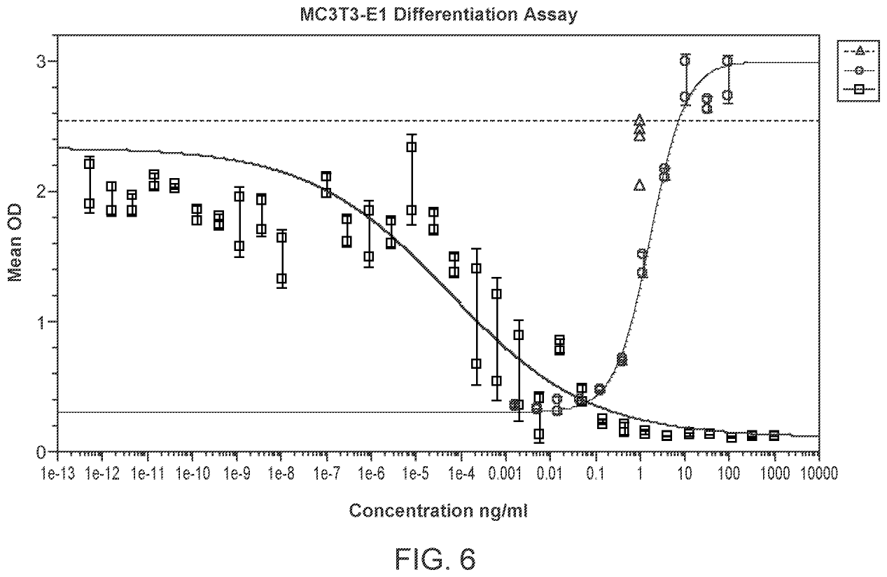

One endogenous protein that is produced by CHO cells that may negatively affect the performance of the target protein is TGFβ1 (Beatson et al. Biotechnol Bioeng 108, 2759-2764 (2011)). TGFβ1 can elicit a biological effect at concentrations in a range as low as femtogram per milliliter (fg/mL) in very sensitive assays like a MC3T3-E1 osteoblast differentiation assay. (See, for example, FIG. 5 and FIG. 6.)

Due to the importance of TGFβ1 to cell proliferation and health, knocking out TGFβ1 in CHO cells was expected to negatively impact the cells (Strutz et al. Kidney Int 59, 579-592 (2001)). For example, it was unpredictable whether the cells would become sick or would exhibit poor growth kinetics compared to wild type cells.

As further described herein, however, knocking out TGFβ1 in CHO cells does allow for the propagation of CHO cells that do not produce TGFβ1. Although slightly slower growth parameters were observed in the knockout cell line, the cell line has been used to successfully produce hWnt3a, hBMP10, and hGDF15 recombinant proteins.

Moreover, in contrast to antibody columns—which were previously used to remove TGFβ1 from compositions including recombinant proteins, using cells that do not produce TGFβ1 allows for a more complete elimination of TGFβ1. In addition, using antibodies to remove TGFβ1 is not effective at removing latent TGFβ1, and latent TGFβ1 can be activated to produce biologically active mature TGFβ1 (Shi et al. Nature 474, 343-349 (2011)).

Furthermore, using a TGFβ1 knockout line to produce a recombinant protein is more cost-efficient than TGFβ1 antibody removal alone because milligrams of an anti-TGFβ1 antibody are otherwise needed to effectively remove TGFβ1.

Thus, in some embodiments, this disclosure describes methods for minimizing the introduction of TGFβ1 into a protein production process by eliminating a cell's ability to produce TGFβ1. Overall, eliminating a cell's ability to produce TGFβ1 provides a lower-cost alternative compared to removal of TGFβ1 with anti-TGFβ1 antibodies, provides for the ablation of both latent and mature TGFβ1 proteins, and results in more consistent removal of TGFβ1 contamination compared to removal with anti-TGFβ1 antibodies alone.

### Compositions

In one aspect this disclosure describes compositions that include a target biologic and very low levels of TGFβ1. Exemplary biologics may include, for example, a recombinant protein (including, for example, a monoclonal antibody, a glycoprotein, a peptide hormone, or a toxin), a ribonucleoprotein, a non-peptide hormone (including, for example, a steroid hormone or an eicosanoid hormone), a glycoprotein, a glycolipid, a subcellular organelle, a blood component, etc. Combinations of the exemplary biologics are also contemplated including, for example, a recombinant glycoprotein or a subcellular organelle isolated from a blood component, etc. In some embodiments, the target biologic includes a biologic drug, that is, a biologic used for treatment of a disease or condition in a subject.

In some embodiments, the composition may preferably be produced by a TGFβ1 knockout cell line including, for example, a TGFβ1 knockout cell line described herein. In some embodiments, a composition produced by a TGFβ1 knockout cell line may be further purified to remove TGFβ1 including, for example, with an anti-TGFβ 1 antibody. In an exemplary embodiment, an antibody column including an anti-TGFβ 1 antibody may be used to remove TGFβ1. An anti-TGFβ1 antibody may be used to remove TGFβ1 introduced during cell culture (including, for example, as a component of fetal bovine serum (FBS)) of the TGFβ1 knockout cell line.

The target biologic may include a protein including, for example, a recombinant protein that has a use in research or therapy. The minimization or elimination of TGFβ1 from a composition including a protein that has a use in research or therapy may be important because TGFβ1 has many functionalities (both desired and unwanted). These functionalities can have important consequences in, for example, immune-oncology and regenerative medicine workflows. See, for example (Glick Cancer Biol Ther 3, 276-283 (2004), Park et al. Cancer Discov 6, 1366-1381 (2016), Tamayo et al. Int J Mot Sci 19, 3928 (2018), Wu et al. Bone Res 4, 16009 (2016)).

In some embodiments, when the target biologic includes a protein, the protein may include a TGFβ superfamily ligand. Exemplary TGFβ superfamily ligands include Wingless-type MMTV Integration Site Family proteins (Wnts), bone morphogenic proteins (BMPs), activins, and growth differentiation factors (GDFs).

The target biologic may be from or derived from any suitable species including, for example, mouse, human, rat, etc.

A Wnt may include, for example, Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, or Wnt16, or a combination thereof. A low level of TGFβ1 contamination in compositions including a Wnt protein may be particularly beneficial because, due to the difficulty of manufacturing recombinant Wnt proteins, the Wnt protein concentration in Wnt protein formulations is generally low. Consequently, users of these low concentration compositions typically have to add a relatively higher volume of the Wnt protein formulation, resulting in the corresponding addition of more contaminating TGFβ1.

A BMP may include, for example, BMP2, BMP3, BMP4, BMPS, BMP6, BMP7, BMP8a, BMP8b, BMP10, BMP11, or BMP15, or a combination thereof. A low level of TGFβ1 contamination in compositions including a BMP is particularly important because while BMPs promote mesenchymal stem cell to osteoblast differentiation, TGFβ1 inhibits osteoblast differentiation. (See, for example, Wu et al. Bone Res. 4:16009 (2016), & Examples 3 and 4.) BMPs have multiple clinical applications. For example, BMP2 and BMP7 are approved for therapeutic clinical use for non-union fracture healing, spinal fusions, and oral surgery.

A GDF may include, for example GDF1, GDF3, GDF5, GDF6, GDF8, GDF9, GDF10, GDF11, or GDF15, or a combination thereof. A low level of TGFβ1 contamination in compositions including a GDF is particularly important to accurately determine the effects of the GDF (Olsen et al. PLoS One 12, e0187349 (2017)).

In some embodiments, the target biologic may include a protein that has a therapeutic use including, for example, an antibody. In some embodiments, the antibody may be a monoclonal antibody. Antibodies may target a protein of an immune checkpoint pathway. For example, exemplary antibodies may target a protein of the CTLA-4 or PD-1 pathway or both. Exemplary antibodies include nivolumab (anti-PD1), pembrolizumab (anti-PD1), atezolizumab (anti-PD-L1), durvalumab (anti-PD-L1), avelumab (anti-PD-L1), tremelimumab (anti-CTLA-4), or ipilimumab (anti-CTLA-4), or combinations thereof, or biosimilars thereof. A low level of TGFβ1 contamination in compositions including an antibody targeting a protein of the PD-1 pathway may be particularly important because TGFβ1-producing cells have been found to upregulate multiple components of the PD-1 signaling pathway, inhibiting antitumor immunity (Park et al. Cancer Discov 6, 1366-1381 (2016)).

Additional exemplary antibodies may include monoclonal antibodies such as bevacizumab, trastuzumab, adalimumab (HUMIRA), infliximab, rituximab, and biosimilars thereof. Other exemplary non-antibody target biologics with therapeutic effects include, for example, etanercept, epoetin alfa, pegfilgrastim, filgrastim, etc.

In some embodiments, the composition includes less than 0.5 ng/mL TGFβ1 protein, less than 0.1 ng/mL TGFβ1 protein, less than 0.01 ng/mL TGFβ1, less than 0.05 ng/mL (50 pg/mL) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 fg/mL) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.

In some embodiments, the composition includes an undetectable level of TGFβ1. In some embodiments, the composition includes an undetectable level of TGFβ1 as measured using the Quantikine ELISA Kit (Catalog No. #DB100B, R&D Systems, Minneapolis, Minn.). The Quantikine ELISA Kit uses a quantitative sandwich enzyme immunoassay technique to detect natural and recombinant TGFβ1 and exhibits a minimum detectable concentration of human TGF-β1 in a range of 1.7 pg/mL to 15.4 pg/mL (mean 4.61 pg/mL). The minimum detectable concentration may be determined by adding two standard deviations to the mean optical density (O.D.) value of twenty zero standard replicates and calculating the corresponding concentration.

In an exemplary embodiment, the composition may include a Wnt or BMP in a range of 0.05 mg/mL to 0.1 mg/mL and TGFβ1 at a concentration of less than 0.02 ng/mL (20 pg/mL). At these levels, the negative effects of TGFβ1 contamination on Wnt's effects in MC3T3-E1 cells are no longer observed in many assays.

In another exemplary embodiment, the composition may include a Wnt or BMP in a range of 0.05 mg/mL to 0.1 mg/mL and TGFβ1 at a concentration of less than 0.001 pg/mL (1 fg/mL) TGFβ1 protein. At these levels, the negative effects of TGFβ1 contamination on Wnt's effects in MC3T3-E1 cells is expected to no longer be observed in the alkaline phosphatase (ALP) activity assay of FIG. 6. (The ND50 of recombinant TGFβ1 in this assay was 53 fg/mL.)

In some embodiments, the composition may be lyophilized. Such compositions may include a buffer, for example, bicarbonate, for reconstitution prior to use or administration, or the buffer may be included in the lyophilized composition for reconstitution with, for example, water. The lyophilized composition can be provided in a syringe, optionally packaged in combination with the buffer for reconstitution, such that the reconstituted composition can be immediately administered to a patient.

In some embodiments, the composition may include at least 40 μg/mL, at least 50 μg/mL, at least 100 μg/mL, at least 150 μg/mL, or at least 200 μg/mL of the target biologic. In some embodiments, the composition may include up to 50 μg/mL, up to 100 μg/mL, up to 150 μg/mL, up to 200 μg/mL, up to 300 μg/mL, or up to 400 μg/mL of the target biologic.

In some embodiments, the composition may be a pharmaceutical composition. Pharmaceutical compositions may be formulated in a variety of forms adapted to the chosen route of administration. The composition will vary depending on mode of administration and dosage unit. For example, for parenteral administration, isotonic saline can be used. For topical administration a cream, including a carrier such as dimethylsulfoxide (DMSO), or other agents typically found in topical creams that do not block or inhibit activity of the peptide, can be used. Other suitable carriers include, but are not limited to alcohol, phosphate-buffered saline, and other balanced salt solutions. The compounds of this invention can be administered in a variety of ways, including, but not limited to, intravenous, topical, oral, subcutaneous, intraperitoneal, and intramuscular delivery. In some aspects, the composition of the present invention may be formulated for controlled or sustained release of the target biologic. In some aspects, a formulation for controlled or sustained release is suitable for subcutaneous implantation. In some aspects, a formulation for controlled or sustained release includes a patch.

In some embodiments, including when the composition is a pharmaceutical composition, the composition may include the target biologic as an active agent and a pharmaceutically acceptable carrier. The active agent may be formulated in a pharmaceutical composition and then administered to a vertebrate, particularly a mammal, such as a human patient, a companion animal, or a domesticated animal, in a variety of forms adapted to the chosen route of administration.

A pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, trehalose, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In some embodiments, the pharmaceutically acceptable carrier includes at least one compound that is not naturally occurring or a product of nature.

### TGFβ1 Knockout Cell Line

In one aspect, this disclosure describes a TGFβ1 knockout cell line and methods of making that cell line.

The cell line may include any suitable cell line. In some embodiments, the cell line may include a CHO cell line. The development and characterization of a TGFβ1 knock out CHO cell line is described in Example 1. Exemplary CHO cell lines include, for example, a CHO-S line, a CHOK1 line, a CHO-DXB11 cell line, and a CHO-DG44 line. In some embodiments, the cell line may include a human embryonic kidney (HEK) cell line including, for example an HEK 293 cell line. In some embodiments, the cell line may include a NS0 cell line. In an exemplary embodiment, the cell line may include a TGFβ1 KO CHO line, as described in Example 1.

The knockout cell line may be made using any suitable means. For example, as described in Example 1, the cell line may be made using CRISPR-based gene editing. In some embodiments, CRISPR-based gene editing may include CRISPR/Cas9-based gene editing. Additionally or alternatively, CRISPR-based gene editing may include CRISPR and a non-Cas9 CRISPR endonuclease including, for example, Cas-CLOVER, MAD7, Cas12a (also known as Cpf1), xCas9, SpCas9-NG, etc. Other gene editing technologies such as TALENs, meganucleases, zinc-finger nucleases, transposons, and homologous recombination may also be suitable to make the knockout cell line.

### Methods of Using a TGFβ1 Knockout Cell Line

A TGFβ1 knockout cell line, as described herein may be used to produce a composition that includes a target biologic and a minimal amount TGFβ1. Because the cell media in which the TGFβ1 knockout cell line is grown may include some TGFβ1, it may not be possible to produce a composition that includes no TGFβ1; however, in a preferred embodiment, the composition produced by the TGFβ1 knockout cell line does not include sufficient TGFβ1 to materially affect the activity or action of the target biologic.

In some aspects, a method for making the composition that includes a target biologic includes culturing a TGFβ1 knockout cell line that overexpresses the target biologic and purifying the target biologic.

The target biologic may be purified by any suitable means. Exemplary methods of purifying recombinant proteins are discussed in, for example, Wingfield, Curr Protoc Protein Sci. 2015; 80: 6.1.1-6.1.35 and Structural Genomics Consortium et al., Nat Methods 5:135-146 (2008). An exemplary method of purifying a monoclonal antibody is described in Corsiero, Mater. Methods 2016; 6:1481.

In situations where a composition produced by the TGFβ1 knockout cell line includes some TGFβ1—including when TGFβ1 is added to the cell culture (as, for example, a component of fetal bovine serum)—purifying the target biologic may include using an anti-TGFβ1 antibody, if needed. In an exemplary embodiment, an antibody column including an anti-TGFβ1 antibody may be used to remove TGFβ1 in the composition including, for example, TGFβ1 introduced during cell culture of the TGFβ1 knockout cell line.

In some embodiments, the TGFβ1 knockout cell line is used to provide a composition that includes less than 0.5 ng/mL TGFβ1 protein, less than 0.1 ng/mL TGFβ1 protein, less than 0.01 ng/mL TGFβ1, less than 0.05 ng/mL (50 pg/mL) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 fg/mL) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.

In some embodiments, the composition includes an undetectable level of TGFβ1. In some embodiments, the composition includes an undetectable level of TGFβ1 as measured using the Quantikine ELISA Kit (Catalog No. #DB100B, R&D Systems, Minneapolis, Minn.). The Quantikine ELISA Kit uses a quantitative sandwich enzyme immunoassay technique to detect natural and recombinant TGFβ1 and exhibits a minimum detectable concentration of human TGF-β1 in a range of 1.7 pg/mL to 15.4 pg/mL (mean 4.61 pg/mL). The minimum detectable concentration may be determined by adding two standard deviations to the mean O.D. value of twenty zero standard replicates and calculating the corresponding concentration.

In some embodiments, the TGFβ1 knockout cell line is used to provide a composition that includes at least 40 μg/mL, at least 50 μg/mL, at least 100 μg/mL, at least 150 μg/mL, or at least 200 μg/mL of the target biologic. In some embodiments, the composition may include up to 50 μg/mL, up to 100 μg/mL, up to 150 μg/mL, up to 100 μg/mL, up to 300 μg/mL, or up to 400 μg/mL of the target biologic. Exemplary ranges of the target biologic include 40 μg/mL to 400 μg/mL; 100 μg/mL to 300 μg/mL; and 40 μg/mL to 100 μg/mL.

In some embodiments, the TGFβ1 knockout cell line may be used to provide a composition having an additional feature or features described in the Compositions section of this disclosure.

### Methods of Using the Compositions

In another aspect, this disclosure describes methods of using a composition produced by a TGFβ1 knockout cell line including, for example, a composition having a feature or features as described in the Compositions section of this disclosure.

In some embodiments, the composition may be used in research. For example, the composition may be used to test the activity of the target biologic. As described in Example 5, a composition produced by a TGFβ1 knockout cell line including Wnt3a as the target biologic may be used in an assay to test the activity of Wnt3a.

In some embodiments, the composition may be used to treat a subject. As used herein, the term “subject” includes, but is not limited to, humans and non-human vertebrates. In preferred embodiments, a subject is a mammal, particularly a human. A subject may be an “individual,” “patient,” or “host.” Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

As used herein “treat,” “treating,” or “treatment” can include therapeutic and/or prophylactic treatments. “Treating a disorder,” as used herein, is not intended to be an absolute term. Treatment may lead to an improved prognosis or a reduction in the frequency or severity of symptoms. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some circumstances, the frequency and severity of symptoms may be reduced to non-pathological levels. In some circumstances, the symptoms of an individual receiving the compositions of the invention are only 90%, 80%, 70%, 60%, 50%, 40%, 30%, 20%, 10%, 5%, or 1% as frequent or severe as symptoms experienced by an untreated individual with the disorder.

The precise dosage and duration of treatment may be a function of the disease being treated and may be determined empirically using known testing protocols or by extrapolation from in vivo or in vitro test data. Concentrations and dosage values may also vary with the severity of the condition to be alleviated. For any particular subject, specific dosage regimens may be adjusted over time according to the individual need and the professional judgment of the person administering or supervising the administration of the compositions.

### EXEMPLARY METHOD ASPECTS

A1. A method for producing a target biologic, wherein the method comprises:
culturing a TGFβ1 knockout cell line that overexpresses the target biologic; and
purifying the target biologic.
   A2. The method of Aspect A1, wherein the method further comprises introducing a vector comprising a gene encoding the target biologic into the TGFβ1 knockout cell line.
   A3. The method of Aspect A1 or A2, wherein the target biologic comprises a recombinant protein.
   A4. The method of any one of Aspects A1 to A3, wherein the target biologic comprises a TGFβ superfamily ligand.
   A5. The method of any one of Aspects A1 to A4, wherein the target biologic comprises a Wnt, a bone morphogenic protein (BMP), an activin, or a growth differentiation factor (GDF), or a combination thereof.
   A6. The method of any one of Aspects A1 to A5, wherein the target biologic comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, or Wnt16, or a combination thereof.
   A7. The method of any one of Aspects A1 to A6, wherein the target biologic comprises BMP2, BMP3, BMP4, BMPS, BMP6, BMP7, BMP8a, BMP8b, BMP10, BMP11, or BMP15, or a combination thereof.
   A8. The method of any one of Aspects A1 to A7, wherein the target biologic comprises GDF1, GDF3, GDFS, GDF6, GDF8, GDF9, GDF10, GDF11, or GDF15, or a combination thereof.
   A9. The method of any one of Aspects A1 to A8, wherein the target biologic is chosen from Wnt3a, Wnt5a, GDF15, BMP4, or BMP2, or a combination thereof.
   A10. The method of any one of Aspects A1 to A9, wherein the target biologic comprises an antibody.
   A11. The method of Aspect A10, wherein the antibody comprises a monoclonal antibody.
   A12. The method of any one of Aspects A1 to A11, wherein the target biologic comprises a biologic drug.
   A13. The method of any one of Aspects A1 to A12, wherein the target biologic comprises a mouse protein.
   A14. The method of any one of Aspects A1 to A12, wherein the target biologic comprises a human protein.
   A15. The method of any one of Aspects A1 to A14, wherein the cell line comprises a Chinese Hamster Ovary (CHO) cell line.
   A16. The method of any one of Aspects A1 to A15, wherein the method further comprises exposing the composition to an anti-TGFβ1 antibody.
   A17. The method of Aspect A16, wherein an antibody column comprises the anti-TGFβ1 antibody.
   A18. A composition comprising the target biologic produced by the method of any one of Aspects A1 to A17.
   A19. The composition of Aspect A18, wherein the composition comprises less than 0.5 ng/mL TGFβ1 protein, less than 0.1 ng/mL TGFβ1 protein, less than 0.01 ng/mL TGFβ1, less than 0.05 ng/mL (50 pg/mL) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 fg/mL) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.
   A20. The composition of Aspect A18 or A19, wherein the composition comprises an undetectable level of TGFβ1.
   A21. The composition of Aspect A20, wherein the level of TGFβ1 is measured using the Quantikine ELISA Kit (Catalog No. #DB100B, R&D Systems, Minneapolis, Minn.).
   A22. The composition of any one of Aspects A18 to A21, wherein the composition comprises at least 40 μg/mL, at least 50 μg/mL, at least 100 μg/mL, at least 150 μg/mL, or at least 200 μg/mL of the target biologic.
   A23. The composition of any one of Aspects A18 to A21, wherein the composition comprises up to 50 μg/mL, up to 100 μg/mL, up to 150 μg/mL, up to 200 μg/mL, up to 300 μg/mL, or up to 400 μg/mL of the target biologic.

### EXEMPLARY COMPOSITION ASPECTS

B1. A composition comprising a target biologic,
wherein the target biologic comprises a TGFβ superfamily ligand, and
wherein the composition comprises less than 0.5 ng/mL TGFβ1 protein, less than 0.1 ng/mL TGFβ1 protein, less than 0.01 ng/mL TGFβ1, less than 0.05 ng/mL (50 pg/mL) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 fg/mL) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.
   B2. A composition comprising a target biologic,
wherein the target biologic comprises a biologic drug, and
wherein the composition comprises less than 0.5 ng/mL TGFβ1 protein, less than 0.1 ng/mL TGFβ1 protein, less than 0.01 ng/mL TGFβ1, less than 0.05 ng/mL (50 pg/mL) TGFβ1 protein, less than 0.02 ng/mL (20 pg/mL) TGFβ1 protein, less than 0.01 ng/mL (10 pg/mL) TGFβ1 protein, less than 0.005 ng/mL (5 pg/mL) TGFβ1 protein, less than 1 pg/mL TGFβ1 protein, less than 0.5 pg/mL (500 fg/mL) TGFβ1 protein, less than 0.01 pg/mL (100 fg/mL) TGFβ1 protein, less than 0.05 pg/mL (50 fg/mL) TGFβ1 protein, less than 0.005 pg/mL (5 fg/mL) TGFβ1 protein, or less than 0.001 pg/mL (1 fg/mL) TGFβ1 protein.
   B3. The composition of Aspect B1 or B2, wherein the composition comprises an undetectable level of TGFβ1.
   B4. The composition of Aspect B3, wherein the level of TGFβ1 is measured using the Quantikine ELISA Kit (Catalog No. #DB100B, R&D Systems, Minneapolis, Minn.).
   B5. The composition of any one of Aspects B1 to B4, wherein the composition comprises at least 40 μg/mL, at least 50 μg/mL, at least 100 μg/mL, at least 150 μg/mL, or at least 200 μg/mL of the target biologic.
   B6. The composition of any one of Aspects B1 to B5, wherein the composition comprises up to 50 μg/mL, up to 100 μg/mL, up to 150 μg/mL, up to 200 μg/mL, up to 300 μg/mL, or up to 400 μg/mL of the target biologic.
   B7. The composition of any one of Aspects B1 to B6, wherein the target biologic comprises a recombinant protein.
   B8. The composition of any one of Aspects B1 to B7, wherein the target biologic comprises an antibody.
   B9. The composition of Aspect B8, wherein the antibody comprises a monoclonal antibody.
   B10. The composition of any one of Aspects B1 to B7, wherein the target biologic comprises a Wnt, a bone morphogenic protein (BMP), an activin, or a growth differentiation factor (GDF), or a combination thereof.
   B11. The composition of Aspect B10, wherein the target biologic comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, or Wnt16, or a combination thereof.
   B12. The composition of Aspect B10, wherein the target biologic comprises BMP2, BMP3, BMP4, BMP5, BMP6, BMP7, BMP8a, BMP8b, BMP10, BMP11, or BMP15, or a combination thereof.
   B13. The composition of Aspect B10, wherein the target biologic comprises GDF1, GDF3, GDFS, GDF6, GDF8, GDF9, GDF10, GDF11, or GDF15, or a combination thereof.
   B14. The composition of Aspect B10, wherein the target biologic is chosen from Wnt3a, Wnt5a, GDF15, BMP4, or BMP2, or a combination thereof.
   B15. The composition of any one of Aspects B1 to B14, wherein the target biologic comprises a mouse protein.
   B16. The composition of any one of Aspects B1 to B14, wherein the target biologic comprises a human protein.
   B17. The composition of any one of Aspects B1 to B16, wherein the target biologic comprises a biologic drug.
   B18. The composition of any one of Aspects B1 to B17, wherein the composition comprises a pharmaceutical composition.

### EXEMPLARY TGFβ1 KNOCKOUT CELL LINE & METHODS OF MAKING ASPECTS

C1. A TGFβ1 knockout cell line, wherein the TGFβ1 knockout cell line comprises a mutation or deletion in the nucleotides encoding TGFβ1.
C2. The TGFβ1 knockout cell line of Aspect C1, wherein the TGFβ1 knockout cell line comprises a Chinese Hamster Ovary (CHO) cell line or a human embryonic kidney (HEK) cell line.
C3. The TGFβ1 knockout cell line of Aspect C1 or C2, wherein the TGFβ1 knockout cell line comprises a stop codon introduced into the nucleotides encoding TGFβ1.
C4. The TGFβ1 knockout cell line of any one of Aspects C1 to C3, wherein the TGFβ1 knockout cell line overexpresses a target biologic.
C5. The TGFβ1 knockout cell line of Aspect C4, wherein the target biologic comprises a TGFβ superfamily ligand, a recombinant protein, a ribonucleoprotein, a non-peptide, a glycoprotein, a glycolipid, a subcellular organelle, a blood component, a biologic drug, or a combination thereof.
C6. A method of making the TGFβ1 knockout cell line of any one of Aspects C1 to C5.
C7. The method of Aspect C6, wherein the method comprises CRISPR-based gene editing.
C8. The method of Aspect C7, wherein the method comprises CRISPR-Cas9-based gene editing.
C9. The method of Aspect C7 or C8, wherein the method comprises CRISPR-based gene editing of the nucleotides encoding TGFβ1.
C10. The method of any one of Aspects C6 to C9, wherein the method comprises introducing a vector comprising a gene encoding a target biologic into the TGFβ1 knockout cell line

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, Mo.) and were used without further purification unless otherwise indicated.

### Example 1

This Example describes the development and characterization of a TGFβ1 knock out cell line.

CRISPR/Cas9 gene editing technology was used to perform directed gene modifications in a host Chinese Hamster Ovary (CHO) cell line. Specific guide RNAs were selected to direct the nuclease activity of the Cas9 enzyme to target known sequences in the host cell genome, “knocking out” the functionality of the CHO TGFβ1 gene, rendering the cells incompetent of producing this protein.

As further described below, the inability of the cells to produce TGFβ1 was confirmed by genomic sequence analysis of individual clones following the gene editing process. Further confirmation was obtained by screening for the presence of TGFβ1 with a Quantikine ELISA kit (R&D Systems, Minneapolis, Minn.).

### Generation of TGFβ1 Knockout (KO) Cell Lines

Knockout of TGFβ1 was performed by B-MoGen Biotechnologies, Inc. (Minneapolis, Minn.) using a gRNA (5′-CAAGACCATCGACATGGAGC-3′) from Synthego Corporation (Menlo Park, Calif.). Clones were isolated and tested for TGFβ1 and TGFβ2 with a Quantikine ELISA kit (R&D Systems, Minneapolis, Minn.). Clones with the lowest level of TGFβ1 were re-isolated. The target region of the target clones was then amplified using PCR, and a clone exhibiting a stop codon in the target region (referred to herein as CHO-S TGFβ1 KO) was selected for further testing.

### Genomic Sequence Analysis

Preparations of CHO-S wildtype (wt) and CHO-S TGFβ1 knockout (KO) genomic DNA were made using Quick-DNA Microprep Kit (Catalog No. D3020, Zymo Research, Irvine, Calif.). PCR was performed on genomic DNA samples to amplify region of interest using GoTaq G2 Hot Start (Catalog No. M7422, Promega, Madison, Wis.).

### PCR Primers:

Forward:
(SEQ ID NO: 4)
5′-GCTCCCCTATTTAAGAACAC-3′
Reverse:
(SEQ ID NO: 5)
5′-GCTCTGCCGGTGGTTTCCTC-3′

### PCR Protocol:

Step 1: 95° C. for 2 minutes

Step 2: 95° C. for 30 seconds

Step 3: 57° C. for 30 seconds

Step 4: 72° C. for 40 seconds

Repeat Step 2 to Step 4 35 times

Step 5: 72° C. for 2 minutes

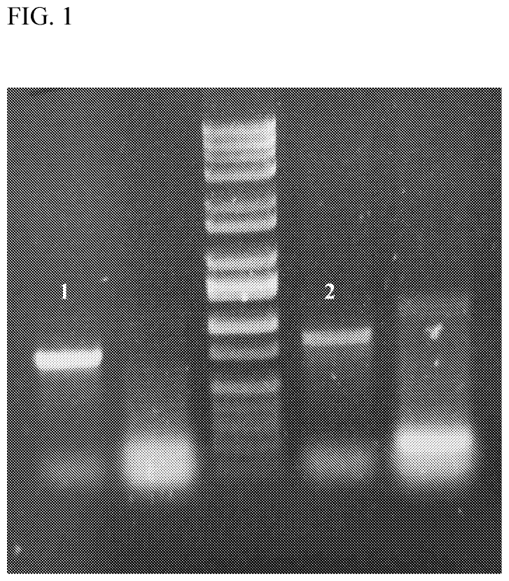

The PCR product was run on a 1% agarose gel and an image was taken on the transilluminator visualizing ethidium bromide staining. Results are shown in FIG. 1 and indicate the introduction of a genetic mutation in the TGFβ1 genomic sequence of the CHO-S TGFβ1 KO cells.

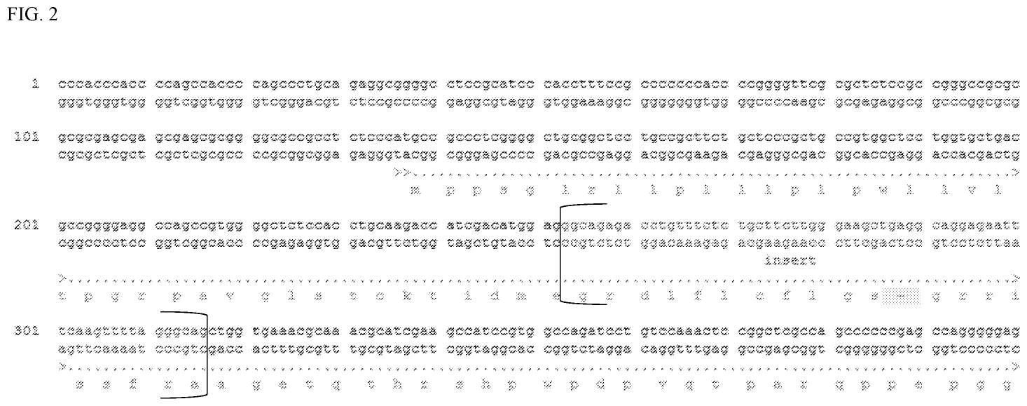

Bands from PCR product of the CHO-S TGFβ1 KO were excised from the gel, purified, and sequenced (Sequencing Primer: 5′-TTCAGGGCTCTCTCCTAACC-3′ (SEQ ID NO:6)). Results are shown in FIG. 2. Sequence analysis confirmed a 64-base pair (bp) insert near the beginning of the TFGβ1 gene, resulting in a stop codon at amino acid 51 introduced by INDELs by the error prone non-homologous end joining (NHEJ) double strand repair mechanism.

### TGFβ1 ELISA Data

CHO-S cells, CHO-S TGFβ1 KO cells, CHO-S TGFβ1 KO cells expressing recombinant hGDF-15, and CHO-S TGFβ1 KO cells expressing recombinant hTGFB1 were plated into EX-CELL ACR CHO Medium (Catalog No. C5467, Sigma Aldrich, St. Louis, Mo.) containing 4 mM L-Glutamine, 1% Pen Strep, and 2.5 mM NaBr and placed in a 33° C. incubator.

HEK293EBNA cells and HEK293EBNA expressing recombinant hGDF-15 were plated into EX-CELL ACR CHO Medium (Catalog No. C5467, Sigma Aldrich, St. Louis, Mo.) containing 4 mM L-Glutamine and 1% Pen Strep placed in a 37° C. incubator.

Conditioned media was collected on day 3 and analyzed using a Human TGFβ1 Quantikine ELISA Kit (Catalog No. #DB100B, R&D Systems, Minneapolis, Minn.).

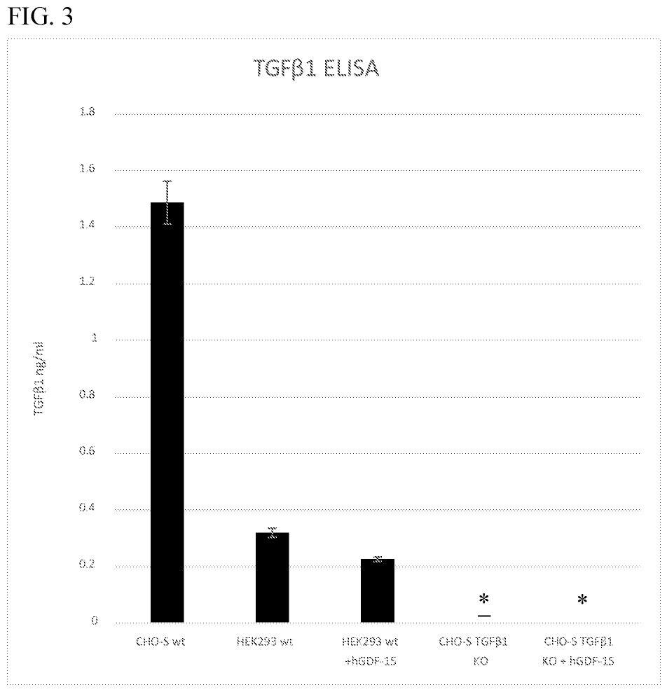

Results are shown in FIG. 3. Detectable amounts of TGFβ1 were present in the conditioned media of wild type CHO-S wt cells, HEK wt cells, and HEK293 expressing recombinant hGDF-15 cells. (FIG. 3 and Table 1.) The amount of TGFβ1 present in the conditioned media of CHO-S TGFβ1 KO samples and CHO-S TGFβ1 KO cells expressing hGDF-15 was below the detectable range. (FIG. 3 and Table 1.) CHO-S TGFβ1 KO cells expressing recombinant hTGFB1 had significantly higher levels of TGFB1 than the parental cell line (Table 1).

TABLE 1

| Sample | ng/mL TGFβ1 |
|---|---|
| CHO-S wt | 1.4850 |
| HEK293 wt | 0.3193 |
| HEK293 wt + hGDF-15 | 0.2260 |
| CHO-S TGFβ1 KO | * |
| CHO-S TGFβ1 KO + hGDF-15 | * |
| CHO-S TGFβ1 KO + hTGFβ1 | 1216.1796 |

*Below detection limit

### Example 2

This Example describes the effect of TGFβ1 contamination on the activation of Smad2 by GDF15 in the DU145 human prostate cancer cell line.

DU145 cells were serum starved for 2 hours prior to adding a protein treatment (with or without TGFβ1 blocking antibody) for 1 hour followed by addition of the hGDF15 protein to the cells for 1 additional hour. The cells were then lysed and analyzing by Western Blot analysis. The antibodies used were a rabbit anti-phospho Smad2 antibody (Cell Signaling Technology, Danvers, Mass.) and a mouse anti-hELF-4E antibody (R&D Systems, Minneapolis, Minn.) (used as a loading control).

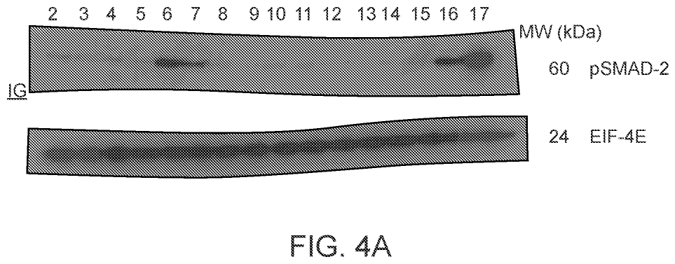

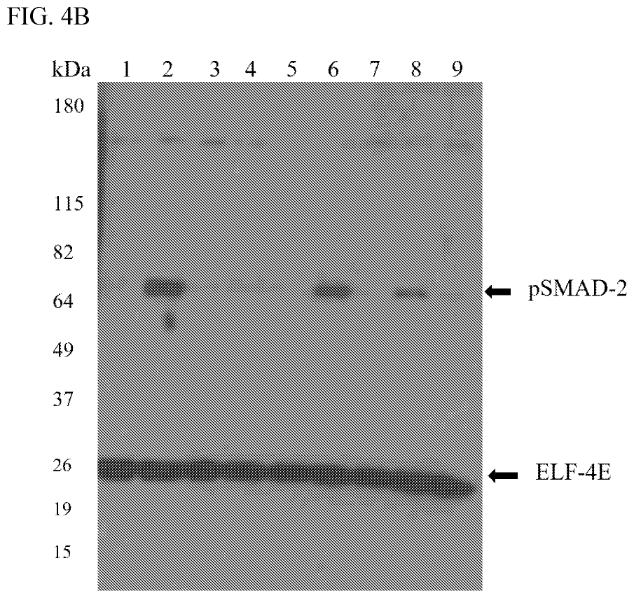

Results are shown in FIG. 4A. Smad2 phosphorylation was detected at a TGFβ1 contamination level of 3.6 pg/mL of media. The contents of each lane of FIG. 4A are described in Table 2A.

Additional results are also shown in FIG. 4B. The contents of each lane of FIG. 4B are described in Table 2B. Smad2 phosphorylation was detected in DU145 cells treated with 2 μg/ml of recombinant human GDF-15 purified from CHO-S wild type cells but not when DU145 cells were treated with 2 μg/ml of recombinant GDF-15 purified from the CHO-S TGFβ1 knockout CHO-S cell line.

TABLE 2A

| | | TGFβ1 | Blocking |
|---|---|---|---|
| | | Protein Levels | TGFβ1 |
| | | in Sample | Antibody |
| | | (pg/μg of | Added |
| Lane | Protein and Dose | protein sample) | (1 μg/ml) |
| 2 | hGDF15 Lot 2 (HEK293) 500 ng/ml | Unknown | No |
| 3 | hGDF15 Lot 2 (HEK293) 250 ng/ml | Unknown | No |
| 4 | hGDF15 Lot 21 (HEK293) 500 ng/ml | 14.31 | No |
| 5 | hGDF15 Lot 21 (HEK293) 250 ng/ml | 14.31 | No |
| 6 | hGDF15 Lot 11 (HEK293) 500 ng/ml | 35.00 | No |
| 7 | hGDF15 Lot 11 (HEK293) 250 ng/ml | 35.00 | No |
| 8 | Untreated Control | 0 | No |
| 9 | Untreated Control | 0 | No |
| 10 | hGDF15 Lot 11 (HEK293) 250 ng/ml | 35.00 | Yes |
| 11 | hGDF15 Lot 11 (HEK293) 500 ng/ml | 35.00 | Yes |
| 12 | hGDF15 Lot 21 (HEK293) 250 ng/ml | 14.31 | Yes |
| 13 | hGDF15 Lot 21 (HEK293) 500 ng/ml | 14.31 | Yes |
| 14 | hGDF15 Lot 2 (HEK293) 250 ng/ml | Unknown | Yes |
| 15 | hGDF15 Lot 2 (HEK293) 500 ng/ml | Unknown | Yes |
| 16 | hTGFβ1 (CHO) 10 ng/ml | (10 ng/ml) | Yes |
| 17 | hTGFβ1 (CHO) 10 ng/ml | (10 ng/ml) | No |

TABLE 2B

| Lane | Contents |
|---|---|
| 1 | Untreated, blank |
| 2 | rhTGFβ1 at 1 ng/mL |
| 3 | rhTGFβ1 at 1 ng/mL + Ch × hTGFβ1 |
| 4 | rhGDF-15 (CHO-s TGFβ1 KO) |
| 5 | rhGDF-15 (CHO-s TGFβ1 KO) + Ch × hTGFβ1 |
| 6 | rhGDF-15 (HEK293EBNA) |
| 7 | rhGDF-15 (HEK293EBNA) + Ch × hTGFβ1 |
| 8 | rhGDF-15 (CHO-s) |
| 9 | rhGDF-15 (CHO-s) + Ch × hTGFβ1 |

### Example 3

This Example describes the effect of TGFβ1 contamination on osteoblast differentiation by Wnt-3a.

Recombinant Mouse Wnt-3a protein from a lot known to have a relatively high level of TGF1 contamination (78 pg of TGFβ1 per microgram (ug) of Wnt-3a protein) resulted in less differentiation of MC3T3-E1 preosteoblasts toward osteoblasts at higher concentrations of Wnt-3a (circles in FIG. 5). When the same dose of mouse Wnt-3a protein was added to MC3T3-E1 preosteoblasts in the presence of a saturating dose (50 ug/mL) of a chicken anti-TGFβ 1 blocking antibody (Catalog No. AF-101-NA, R&D Systems, Minneapolis, Minn.), a rescue of osteoblast differentiation was observed with alkaline phosphatase activity rebounding to the predicted plateau. Results are shown in FIG. 5.

These results demonstrate that neutralizing TGFβ1 in purified mouse Wnt-3a protein removes a negative regulator of osteoblast differentiation.

### Example 4

This Example describes the effect of TGFβ1 on a Wnt-3a-mediated increase in alkaline phosphatase (ALP) expression.

MC3T3/E1 osteoblast cells were seeded overnight at 1×10⁴ cells/well. The next day, media from was removed (leaving the MC3T3/E1 osteoblast cells on the plate), and new media including Wnt-3a or Wnt-3a and TGFβ1 was added. The cells were incubated for 3 additional days in the presence of Wnt-3a or Wnt-3a and TGFβ1, and alkaline phosphatase (ALP) activity was measured (Pacifici et al., Exp. Cell Res. 1991; 195, 38-46). Results are shown in FIG. 6.

MC3T3-E1 preosteoblasts treated with recombinant human Wnt-3a protein alone exhibited dose-responsive induction of osteoblast differentiation, signified by increasing ALP activity three days after addition of the Wnt-3a protein. (FIG. 6, circles).

When a constant dose of 20 ng/mL of recombinant human Wnt-3a protein was added to MC3T3-E1 cells for three days, a relatively high level of ALP activity resulted. (FIG. 6, flat line and triangles.) If the same steady dose of 20 ng/mL of recombinant human Wnt-3a protein was added to cells and a dose titration of recombinant human TGFβ1 was added (FIG. 6, squares), inhibition of Wnt-3a-mediated osteoblast differentiation was observed at a neutralizing dose of 50% (ND50) of 53.3 fg/mL TGFβ1.

### Example 5

This Example describes the effect of TGFβ1 contamination of human Wnt3a (hWnt3a) purified from CHO wt and CHO TGFβ1 KO cell lines in assays designed to measure the effects of hWnt3a.

A CHO TGFβ1 KO cell line was prepared as described in Example 1. All CHO-S or CHO-S TGF1 KO lines expressing hWnt3a were made by transfection of a DNA expression plasmid expressing hWnt3a and selected with puromycin to select for clonal stably integrated cell lines overexpressing hWnt3a. Recombinant human Wnt3a purified from CHO-S wt cells stably expressing hWnt3a shows relatively high levels of TGFβ1 protein compared to reduced levels of TGFβ1 protein observed in hWnt3a isolated from a TGFβ1 knockout CHO-S cell line that stably overexpresses hWnt3a (CHO-S TGFβ1 KO+Wnt3a). Results are shown in Table 3. Lot #RSK69 had 165 pg TGFβ1 protein per μg of hWnt3a protein and lot #RSK51 had 114.6 pg TGFβ1 protein per μg of hWnt3a protein. In contrast, hWnt3a protein purified from the CHO-S TGFβ1 KO line (lot #DLGC02) had 4.97 pg TGFβ1 protein per μg of hWnt3a protein. The low level of TGFβ1 protein in the hWnt3a protein purified from the TGFβ1 KO line is due to the use of 2% fetal bovine serum (FBS) included in the cell culture media; including FBS is necessary to culture the cells which produce active recombinant Wnt proteins. The source of this TGFβ1 protein is not cell-derived but from serum used during cell culture.

TABLE 3

| | | | TGFβ1 Levels | |
|---|---|---|---|---|
| | | | (per microgram | |
| Cell Line | Protein | Lot # | hWant3a) | |
| CHO-S wt + hWnt3a | hWnt3a | RSK69 | 165 | pg |
| CHO-S wt + hWnt3a | hWnt3a | RSK51 | 114.6 | pg |
| CHO-S TGFβ1 KO + hWnt3a | hWnt3a | DLGC02 | 4.97 | pg |

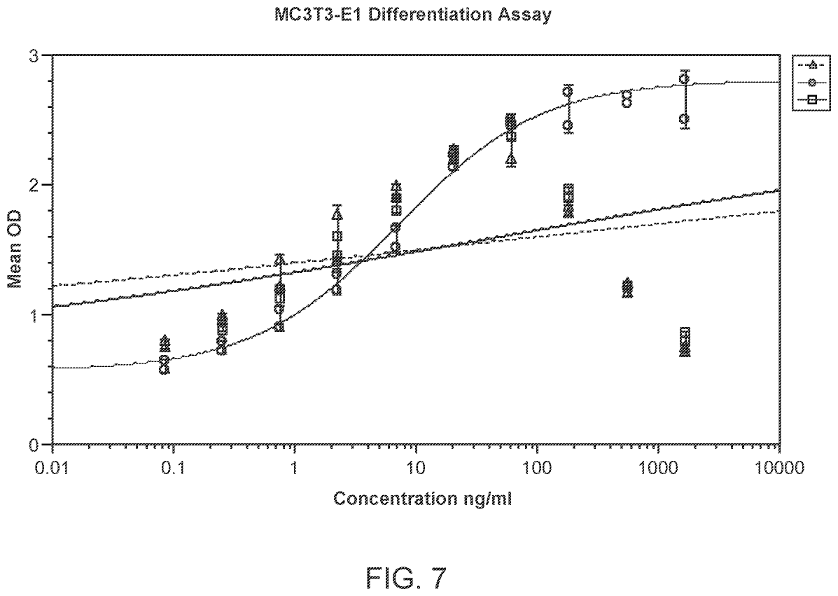

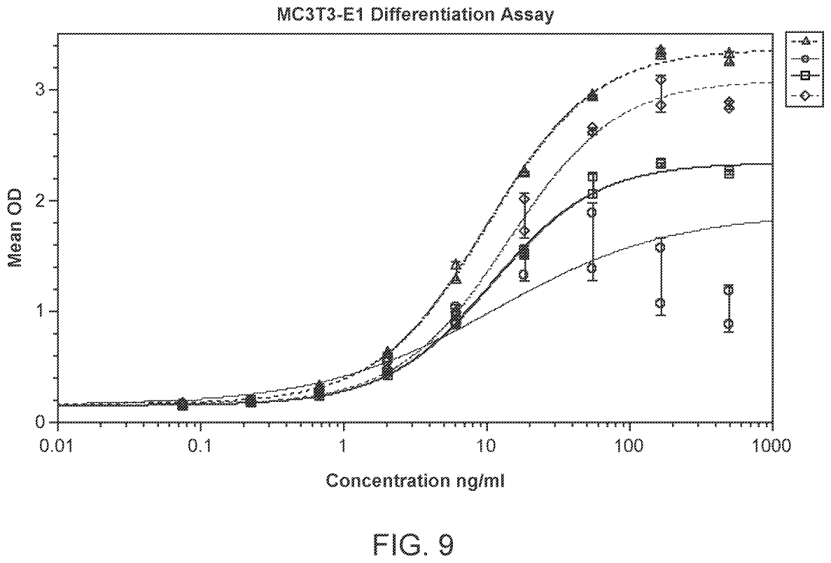

The hWnt3a protein from lots purified from wild type CHO-S cells showed inhibitory effects at the highest levels of hWnt3a treatment in a MC3T3-E1 osteoblast differentiation assay (FIG. 7 & FIG. 9). Reduced osteoinductive activity was observed at higher doses of hWnt3a (1.66 μg/ml, 0.556 μg/ml, and 0.185 μg/ml) purified from wild type CHO-S cells but was not observed when treating the MC3T3-E1 cells with hWnt3a derived from CHO-S TGFβ1 KO cells (FIG. 7 & FIG. 9). The “dip” in activity at higher doses of Wnt3a protein is due to TGFβ1 contamination and can be reversed with a TGFβ1 function blocking antibody (see, e.g., FIG. 5 & FIG. 9). Moreover, as shown in FIG. 9, hWnt3a protein with high levels of TGFβ1 contamination results in lower osteo-inductive activity at high hWnt3a doses in a MC3T3-E1 assay than hWnt3a protein purified from TGFβ1 KO CHO-S cells or hWnt3a from CHO-S TGFβ1 KO cells.

The recombinant hWnt3a purified from CHO-S TGFβ1 KO cells showed substantially lower levels of TGFβ1 compared to hWnt3a purified from CHO-S wt cells (see Table 3). This low level of TGFβ1 contamination in recombinant hWnt3a purified from CHO-S TGFβ1 KO cells arises from the 2% serum that is necessary during cell culture to produce active Wnt proteins. Addition of the TGFβ1 blocking antibody rescued the inhibitory effects of the lower level TGFβ1 contamination arising from the serum in CHO-S TGFβ1 KO cells expressing hWnt3a (FIG. 9). These data demonstrate that hWnt3a purified from CHO-S TGFβ1 KO cells contains significantly less TGFβ1 contamination compared to hWnt3a purified from CHO-S wt cells expressing hWnt3a. In addition, these data further demonstrate that the inhibitory effects on MC3T3-E1 osteoblast differentiation correlates with relative amounts of TGFβ1 contamination in purified hWnt3a proteins.

Interestingly, TGFβ1 contamination did not affect all assay systems. For example, no major functional differences between hWnt3a proteins purified from CHO-S wt and CHO-S TGFβ1 KO cells were detected in in a HEK293 Wnt reporter assay, as further described below.

A stable cell line was generated to incorporate nine T cell factor (TCF) binding elements upstream of a SEAP reporter gene (Korinek et al. Science 275, 1784-1787 (1997)). Wnt proteins bind to Wnt receptors on the cell surface of this HEK293 cell line resulting in a cascade of intracellular events culminating in β-catenin binding to TCF transcription factors resulting in production of the reporter gene SEAP. SEAP is secreted into the media and SEAP activity assays provide an indication of Wnt pathway activity. Recombinant TGFβ1 protein has been tested in this Wnt reporter assay previously with no activation of this Wnt specific assay.

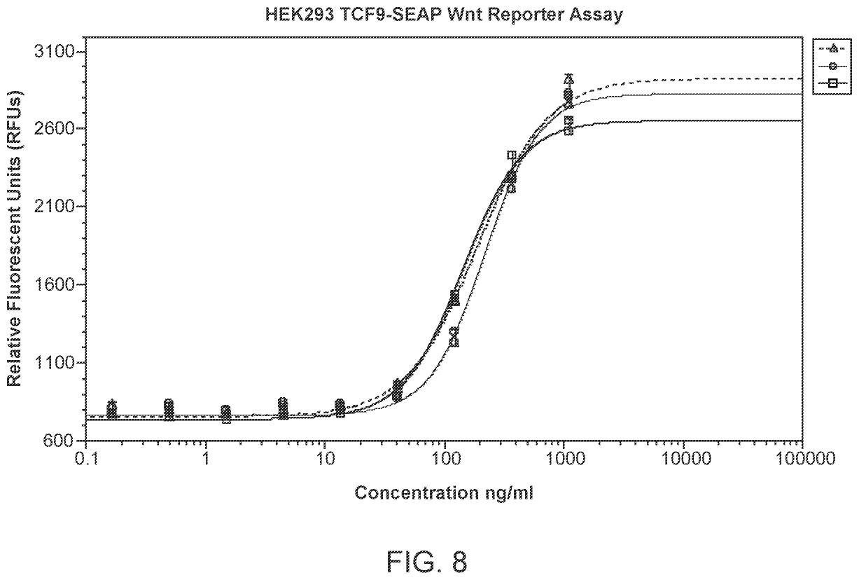

Results are shown in FIG. 8. These results were not surprising; titrations of TGFβ1 protein in this HEK293 Wnt reporter have not been shown to have any effect in this Wnt reporter system. These data demonstrate that TGFβ1 contamination is cell- and assay-context dependent, and not all biological activity assays will show sensitivity to TGFβ1 contamination.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for producing a target biologic, wherein the method comprises:
culturing a TGFβ1 knockout cell line that overexpresses the target biologic; and
purifying the target biologic.

2. The method of claim 1, wherein the method further comprises introducing a vector comprising a gene encoding the target biologic into the TGFβ1 knockout cell line.

3. The method of claim 1, wherein the target biologic comprises a recombinant protein.

4. The method of claim 1, wherein the target biologic comprises a TGFβ superfamily ligand.

5. The method of claim 1, wherein the target biologic comprises an antibody.

6. The method of claim 1, wherein the target biologic comprises a mouse protein or a human protein.

7. The method of claim 1, wherein the cell line comprises a Chinese Hamster Ovary (CHO) cell line.

8. A composition comprising the target biologic produced by the method of claim 1, wherein the composition comprises less than 0.5 ng/mL TGFβ1.

9. A composition comprising the target biologic produced by the method of claim 1, wherein the composition comprises less than 0.5 fg/mL TGFβ1.

10. The composition of claim 8, wherein the composition comprises an undetectable level of TGFβ1.

11. A composition comprising a target biologic,
wherein the target biologic comprises a TGFβ superfamily ligand, and
wherein the composition comprises less than 0.5 ng/mL TGFβ1.

12. A composition comprising a target biologic,
wherein the target biologic comprises a biologic drug, and
wherein the composition comprises less than 0.5 ng/mL TGFβ1.

13. The composition of claim 11, wherein the composition comprises less than 0.5 fg/mL TGFβ1.

14. The composition of claim 11, wherein the composition comprises an undetectable level of TGFβ1.

15. The composition of claim 11, wherein the target biologic comprises a recombinant protein.

16. The composition of claim 11, wherein the target biologic comprises a Wnt, a bone morphogenic protein (BMP), an activin, or a growth differentiation factor (GDF), or a combination thereof.

17. The composition of claim 11, wherein the target biologic comprises an antibody.

18. The composition of claim 8, wherein the target biologic comprises a mouse protein or a human protein.

19. The composition of claim 8, wherein the target biologic comprises a biologic drug.

20. The composition of claim 8, wherein the composition comprises a pharmaceutical composition.
